(19) United States
(12) Patent Application Publication
(10) Pub. No.: US 2002/0151858 A1
(43) Pub. Date: Oct. 17, 2002

(54) Absorbent articles for various torso sizes

(76) Inventors: Hamzeh Karami, Lockhaven, PA (US); Kambiz Damaghi, Kings Point, NY (US); Babak Damaghi, Kings Point, NY (US)

Correspondence Address: Stoll, Miskin, Hoffman & Badie, The Empire State Building, Suite 6110, 350 Fifth Avenue, New York, NY, 10118, US

(21) Appl. No.: 10/134,346

(22) Filed: Apr. 26, 2002

Related U.S. Application Data
(63) Continuation-in-part of application No. 09/544,092, filed on Apr. 6, 2000.
Application No. 09/544,092 is a continuation-in-part of application No. 09/376,282, filed on Aug. 18, 1999.
Application No. 09/376,282 is a continuation-in-part of application No. 09/149,265, filed on Sep. 8, 1998, now Pat. No. 6,306,121.
Application No. 09/149,265 is a continuation-in-part of application No. 09/097,198, filed on Jun. 12, 1998, now Pat. No. 6,413,249.

(51) Int. Cl. A61F 13/15

(52) U.S. Cl. 604/385.3; 604/389; 604/392

(57) ABSTRACT

A disposable absorbent article is provided to fit various torso sizes. The article having a laterally extending belt, which may be partly stretchable or elasticated, and an insert member disposed vertically relative to the belt. The insert member has a proximal end for attachment to the belt, and a distal end having two spaced apart tape tab fasteners. The belt has two wing portions disposed to the left and right sides of a vertical axis running through the insert member, and is provided with tape tab fasteners and landing zones adapted to engage with each other and with the tape tab fasteners on the distal end of the insert member when the belt is wrapped around the torso of a wearer and the insert member is folded onto the belt. In one embodiment, one wing is longer than the other wing of the belt, and in a further embodiment the belt is a half-belt, or one wing, attached to the left or right side of the proximal end of the insert member. Several fastening systems are provided for fastening the absorbent article when it is assembled for wear.

### RELATED APPLICATIONS

This application is a continuation-in-art of application Ser. No. 09/544,092 filed Apr. 6, 2000, which is, in turn, a continuation-in-part of application Ser. No. 09/376,282 filed Aug. 18, 1999, which is, in turn, a continuation-in-part of application Ser. No. 09/149,265 filed Sep. 8, 1998, which is, in turn, a continuation-in-part of application Ser. No. 08/097,198 filed Jun. 12, 1998.

### FIELD OF THE INVENTION

The present invention relates generally to absorbent articles such as disposable diapers, infant training pants, adult undergarments and incontinence briefs used for absorption and containment of urine and other body exudates. In one aspect, the present invention relates to such absorbent articles which assure improved body fit and prevent leakage of fluids and body exudates during their use. In a more particular aspect, the absorbent articles of the present invention are designed to fit comfortably about different size torsos, easy to wear, change and remove, less bulky than the prior art type absorbent articles, and which can be more readily mass produced economically.

### BACKGROUND OF THE INVENTION

Ever since infant diapers and adult incontinence briefs began to find widespread institutional and household use, more and more attention has been focused on improving body fit and comfort, leakage prevention, ease of wear and removal, and simplicity and economy of their mass production. In case of adult incontinent persons, varying hip, waist and torso sizes often dictates the relative dimensions and different constructions of the brief with a view to assuring fitness, comfort and leak prevention. In addition, the simplicity of such varied size briefs plays a major practical role from the standpoint of economy of their production and utilization.

Current disposable briefs are frequently oversized in order to fit persons with pre-determined hip and waist girth sizes. For example, a medium size brief is usually designed for persons having a waist-hip size of 32-44 inches. These briefs are provided with adhesive tabs to secure the brief on the body of the wearer. Since these briefs are intended to be worn by persons having different girth sizes of about 32 to 42 inches, they are provided with conventional tape tabs and landing zones in order to assure satisfactory and comfortable fit. For persons with larger girth sizes, more material is required to form the brief than is necessary, thus contributing to waist in economy of fabrication. The problem is even more acute when forming extra-large briefs for persons with girth sizes of 56-64 inches.

Although most present-day briefs contain copious amounts of super-absorbent polymer (SAP) and fluff material, the user still experiences fluid leak and discomfort. The introduction of so-called “pull-up”, also known as “pull-on” type briefs in recent years was designed to alleviate some of the problems, however, these briefs are often difficult to put on an incontinent person. Even more difficulty is experienced when removing pull-up briefs when they have been soiled. In order to assure body tight fitness, the prior art briefs employ hook and loop fastening systems comprising hooks and loops wherein the hooks are defined by tape tab-like structures and the loops are defined by special substrates defining landing zones onto which the hooks can be securely engaged. Ordinarily, a plurality of complementary hooks and loops are needed to afford a tight fit around the torso. The material used for the loop component of the fastening system, i.e., the loop type landing zones, are so expensive that makes it cost prohibitive to manufacture baby diapers, and are even more cost prohibitive for making adult briefs since they require the use of more landing zone materials.

In the aforementioned commonly assigned application Ser. No. 09/376,282 there is described a generally T-shaped brief and L-shaped brief designed to provide greater degree of fluid leak prevention while reducing the material required to fabricate the brief. The brief described therein is generally one-piece integral disposable article having a cross-piece and an elongated member which, in a stretched view, displays a generally T-shaped, or L-shaped configuration. The briefs described in this application constitute improvement over the prior art briefs, including those described in the aforementioned application.

Therefore, it is an object of this invention to provide baby diapers and adult incontinent briefs with easy to use fastening systems which assures tight body fit for various torso sizes, at low cost.

It is another object of this invention to provide such diapers, briefs and other absorbent garments for use by incontinent persons which are provided with unique waist belt and fastening systems such as hook and loop or other combinations of male and female fasteners designed to assure maximum fit comfort and prevention of leakage.

It is a further object of this invention to provide diapers, briefs and absorbent articles of the types herein described having uniquely designed and arranged complementary male and female fastening systems which minimize the need to use large quantities of male and female fastening system, and hence afford more economical means of manufacturing these absorbent articles.

It is still an object of this invention to provide such absorbent articles which can be readily put on by an incontinent person with minimal discomfort and inconvenience, and which can be removed when soiled with the problems normally experienced in case of putting on or removing soiled briefs of the prior art variety.

The foregoing and other objects and advantageous features of the absorbent articles of the present invention will be more fully appreciated from the following detailed description and the accompanying drawings.

### SUMMARY OF THE INVENTION

The disposable absorbent article of the present invention is designed to fit snugly around different individual torso sizes. It comprises a combination of a partially or fully elasticated belt and an insert member, which together can form an integral article, or they may be separate pieces adapted to be used together in the same manner as the integral article. In flat out position, the belt comprises two lateral ends and an insert member vertically disposed relative to the belt wherein said insert member has a proximal end attached to said belt, and a distal end adapted to be folded onto said proximal end and attached thereto. The belt and the insert member are provided strategically positioned male and female fasteners so that when the article is worn the fasteners engage to one another to firmly and comfortably secure the article around the waist of the user.

The fasteners which are employed may be tape tabs and landing zones, and male and female fasteners such as the four fastening systems described in copending application Ser. No. 09/844,726 filed Apr. 27, 2001, which is continuation-in-part of application Ser. No. 09/797,334 filed Mar. 1, 2000, the disclosures of which are fully incorporated herein by reference. In one construction, the belt is provided with a male fastener (e.g., hook) at one lateral end, and a female fastener (e.g., loop) at the other lateral end, and two spaced apart female fasteners (e.g., loops) disposed between the proximal end of said insert member and the hook disposed at the said lateral end of the belt. Two spaced apart male fasteners (e.g., hooks) are disposed at the distal end of said insert members such that when the lateral ends of said belt are wrapped around the torso of the user, said male fasteners on said distal end of said insert member engage with said two female fasteners on the belt.

The disposable absorbent article of the present invention can be formed using minimal quantity of expensive materials. The insert member is usually formed of various layers as in the prior art absorbent articles. The relative locations of the fasteners may be changed without changing the construction or use of the absorbent article. For example, instead of using male fasteners at the distal end of the insert member, female fasteners may be used, in which case, the two female fasteners between the proximal end of the insert member and the end male fastener may be replaced with two male fasteners adapted to engage with the respective female fasteners at the distal end of the insert members.

### BRIEF DESCRIPTION OF THE DRAWINGS

In the drawings, wherein like reference numerals are employed to designate like parts:

FIG. 1 is a plan view of the diaper of this invention in a fully stretched position;

FIG. 2 is a perspective view of the diaper illustrated in FIG. 1;

FIG. 3 is a perspective view of the diaper shown in FIG. 2 in assembled position;

FIG. 4 is a stretched plan view of the belt component of the diaper of this invention wherein a portion of the belt is elasticated by a plurality of spaced apart, generally parallel elastic elements;

FIG. 5 is a sectional view taken along the line 5-5 of FIG. 4;

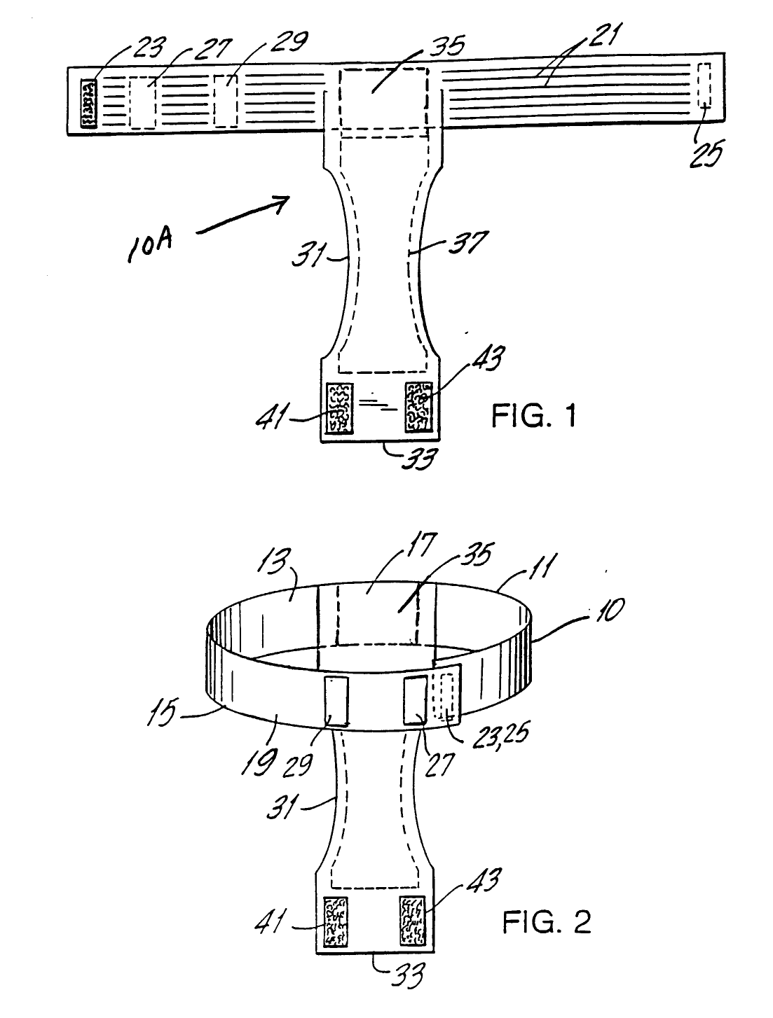

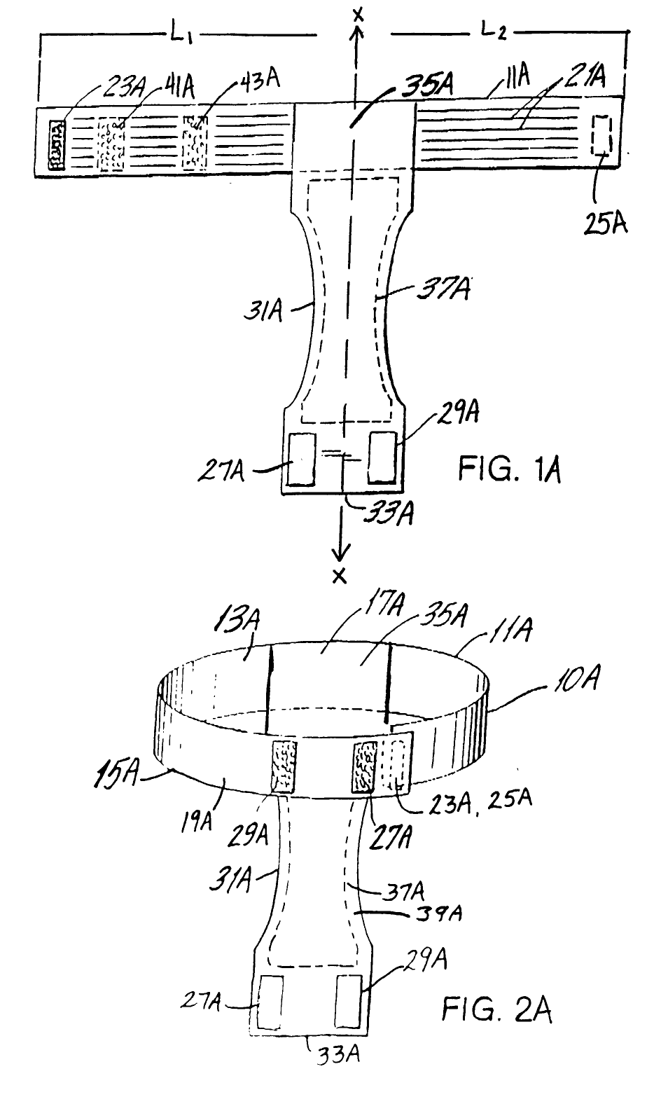

FIG. 1A is a plan view of a diaper similar to FIG. 1 but showing different locations of the fasteners on the belt and the insert members, and wherein the belt has a shorter wing on one side of the insert member than the wing on the other side of the insert member;

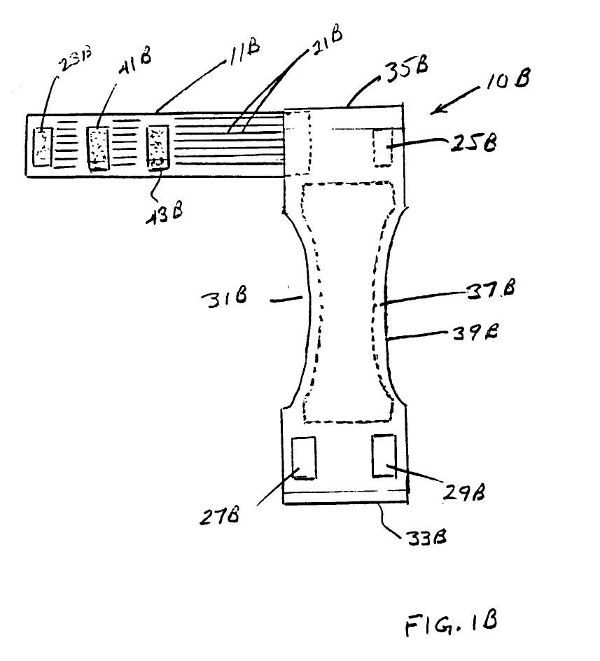

FIG. 2A is a perspective view of the diaper shown in FIG. 1A;

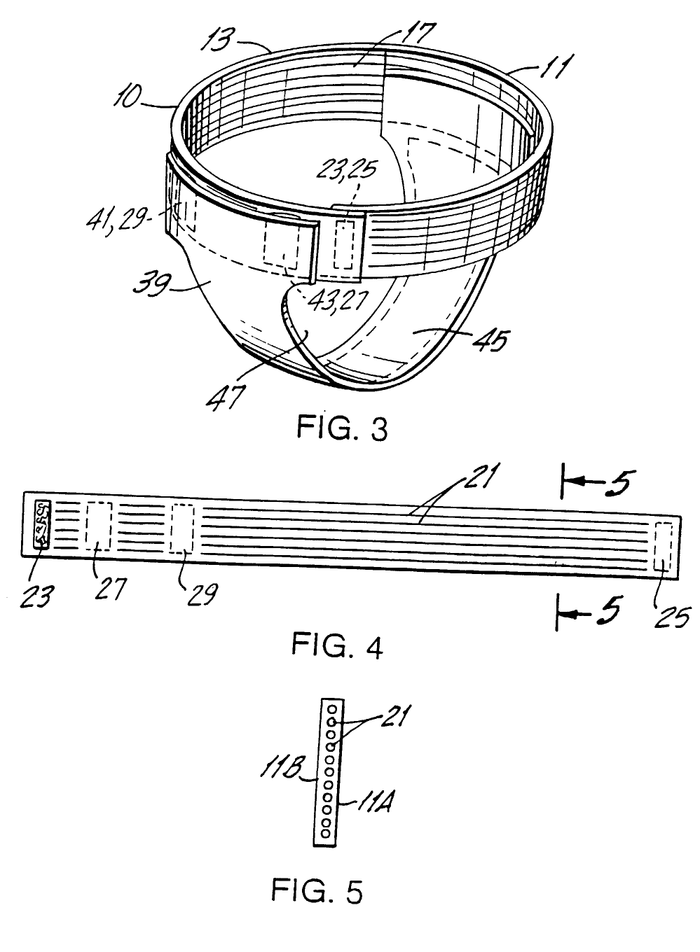

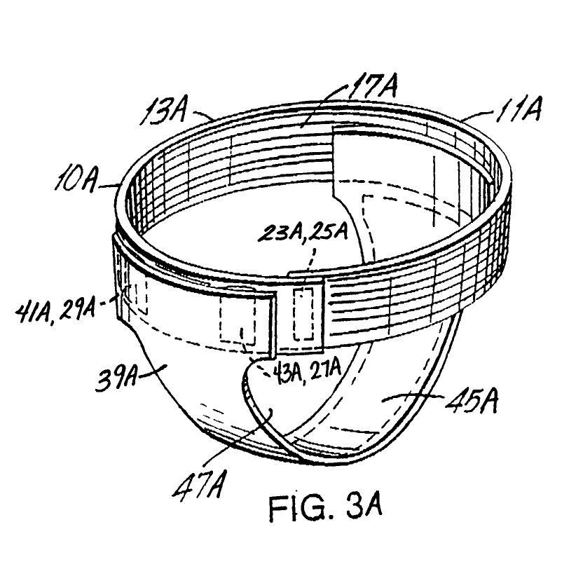

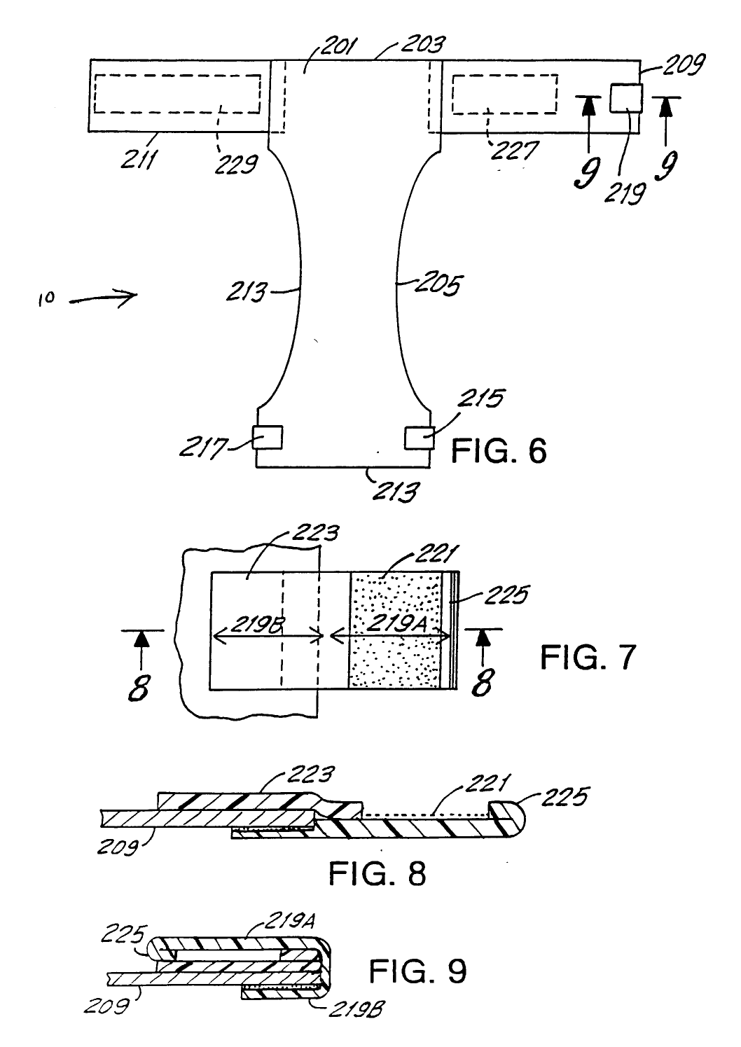

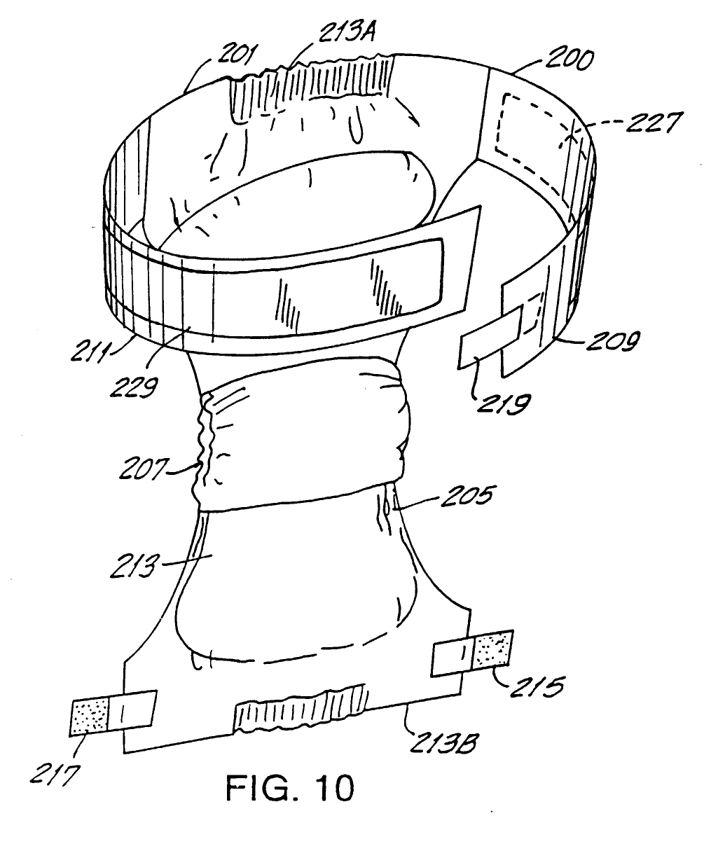

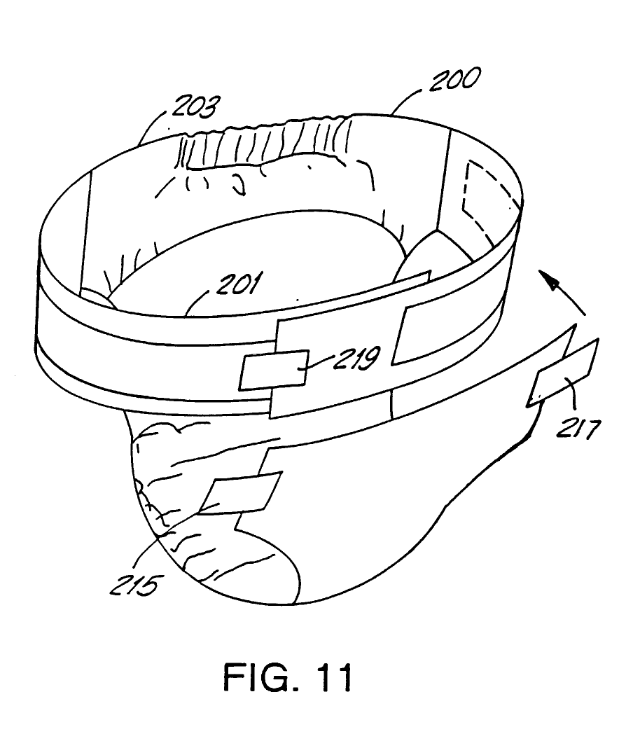

FIG. 3A is a perspective view of the diaper shown in FIG. 2A in assembled position;

FIG. 1B is a stretched plan view of a diaper according to another embodiment of the invention consisting of a single wing or belt (half-belt) attached to an insert member;

FIG. 6 is a view of a T-shaped diaper in stretched position employing a tape tab and landing zone fastening system according to one embodiment of the present invention;

FIG. 7 is a top view of the tape tab shown in FIG. 6, with the fastener tape in open, ready to be used position;

FIG. 8 is a sectional view taken along the line 8-8 of FIG. 7;

FIG. 9 is a sectional view taken along the line 9-9 of FIG. 6;

FIG. 10 is a partly perspective view of a diaper having a tape tab and landing zone fastening system ready for use;

FIG. 11 is a perspective view of the diaper shown in FIG. 10 in ready to be assembled position;

FIG. 12 is a view similar to FIG. 6 employing multi-layer tape tab and landing zone fastening system according to another embodiment of the present invention;

FIG. 13 is a top view of the multi-layer tape tab of FIG. 12 with the fastener tape in open ready to use position;

FIG. 14 is a sectional view taken along the line 14-14 of FIG. 13;

FIG. 15 is a sectional view taken along the lines 15-15 of FIG. 12;

FIG. 16 is a stretched view of a T-shaped diaper similar to FIGS. 11 and 12 but employing a hook and loop fastening system according to a different embodiment of the present invention;

FIG. 17 is a top view of the hook system employed in FIG. 16 with the tape tab in open ready to be used position;

FIG. 18 is a sectional view taken along the line 18-18 of FIG. 17;

FIG. 19 is a sectional view taken along the line 19-19 in FIG. 16;

FIG. 20 is a view FIG. 20 is a view similar to FIG. 16 but employs a multi-layer hook and loop fastening system according to yet another embodiment of present invention;

FIG. 21 is a top view of the multi-layer hook and loop fastening system used in FIG. 20 with the tape tab in open ready to be used position;

FIG. 22 is a sectional view taken along the line 22-22 in FIG. 21;

FIG. 23 is a sectional view taken along the line 23-23 in FIG. 20, and

FIG. 24 is a view similar to FIG. 10 employing a multi-layer hook and loop fastening system, and illustrates the diaper after the first attempt of fastening the tape system and peeling open the diaper to show the tape tabs as in FIG. 22.

### DETAILED DESCRIPTION OF THE PREFERRED EMBODIMENT OF THE INVENTION

In the ensuing detailed description, the present invention will be described by reference to a diaper and specifically illustrated as a generally T-shaped diaper. However, it must be understood that this description is applicable to like absorbent articles such as incontinence briefs, training pants and the like articles of the same or different configuration.

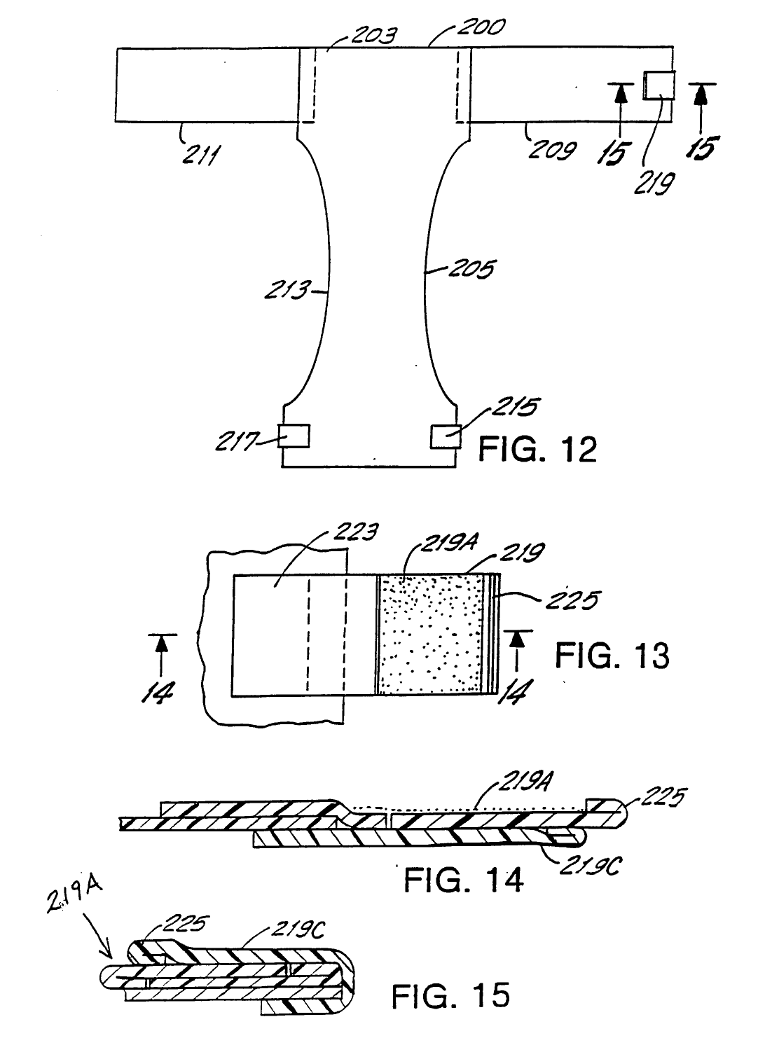

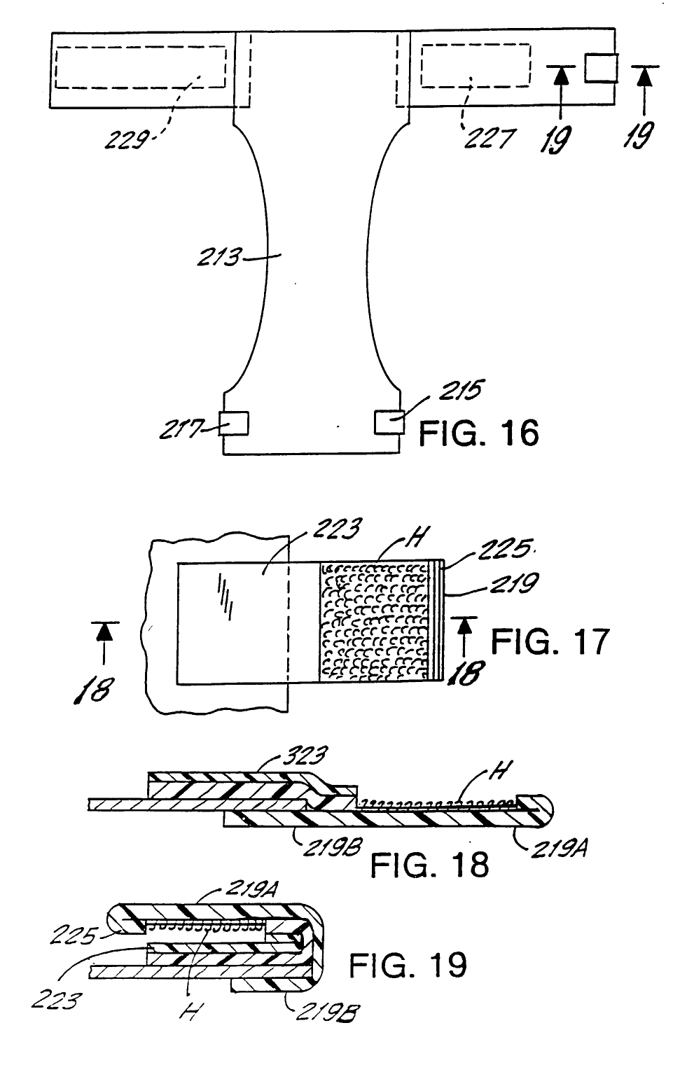

Thus, referring to FIGS. 1-3, the diaper of this invention is designated by 10 and comprises an elasticated belt member 11 adapted to be wrapped around the waist of the wearer. The belt 11 has a back waist portion 13 and a front waist portion 15, each comprising a bodyside surface 17 and an opposed surface 19 away from the bodyside of the wearer. The belt 11 is made of spunbond nonwoven stretchable material. Thus, the belt 11 is made stretchable by a plurality of spaced apart, generally parallel elastic elements or strings 21 (see FIG. 4) running in the cross (horizontal) direction, secured between two layers of nonwoven. Alternatively, the belt 11 may be made from an elastomeric material which itself is inherently elastic, in which case no elastic elements are needed for imparting elasticity to the belt. Such elastomeric composite materials are well known in the art, including natural and synthetic rubbers, polyurethane and polyethylene. In its preferred embodiment, the belt 11 exhibits elasticity which varies from about 50 to about 500, preferable from about 100 to about 300 percent. A preferred composite elastic material for belt is Fabriflex 204 series made by Tredegar Film Products, Richmond, Va. 23225. When a belt of such degree of elasticity is used, less of the expensive nonwoven material will be required to form the diaper, with consequent reduction in cost and economy of manufacture. In addition, one size product can be made to fit persons of different hip/waist torso sizes.

The body side surface 17 of the belt 11 is provided with a male fastener, e.g., hook 23 attached at one end of the belt, and a female loop 25 is disposed at the other end of the body side surface 19, as shown in FIGS. 1 and 4. A pair of spaced apart, generally parallel disposed loops 27,29 are located on the opposed surface 19 of the belt and are adapted to provide female receiving zones when the belt 11 is wrapped around the waist.

The diaper 10 also comprises an insert 31, having a free distal end 33 away from the belt 11 when the diaper is viewed in the stretched flat position, and a proximal end 35 which is attached to the belt 11 using conventional adhesives, heat, ultrasonic energy or other known conventional methods. The insert 31 is similar to conventional disposable briefs and comprises a coversheet, a backsheet and an absorbent core disposed between the coversheet and the backsheet. The materials or nonwoven films used in making the diapers of this invention are, generally, of type and variety known in the prior art as, for example, described in the aforementioned copending application Ser. No. 09/149,265, the disclosure of which is incorporated herein by reference. Thus, the coversheet may be made of a liquid pervious, soft compliant material which is non-irritating and is skin friendly. By way of examples, such materials include porous foams, reticulated foams, plastics, natural fibers such as woods or cotton fibers, synthetic fibers made of polyester or poly-propylene available from First Quality Fibers, Inc., McElhattan, Pa., or made from a suitable combination of said materials.

The absorbent core may be formed from a wide variety of liquid absorbent materials of the type used in making absorbent disposable diapers and other absorbent articles. This core may be made of wood pulp fibers and super-absorbent polymers (SAP) such as IM 2000 series available from BASF Corporation. Alternatively, the absorbent core may be made of dual construction, in which case, the SAP may be placed between each layer of the absorbent material. The amount of SAP in the SAP-wood pulp fiber mixture may be from about 5 to about 60 weight percent based on the total weight of the mixture.

The backsheet is preferably a composite made of spunbond nonwoven and a polyethylene film. In some instances, it is desired to provide a film backing, usually a polyethylene film which is liquid, air and preferably vapor impermeable. This film backing is placed under the absorbent core in order to prevent the fluid and body exudates from leaking and otherwise soiling the user's clothing or bed. Polyethylenes suitable as backing film are available from Clopay Plastics, Cincinnati, Ohio.

It may be further desired to include an acquisition layer in order to control absorption and distribution of the fluid and body exudates. The acquisition layer is usually made of chemically bonded nonwoven made of polyester, rayon or polypropylene available from American Nonwovens, Columbus, Mo.

It must be understood that the aforementioned descriptions of the various layers which form the insert 31 do not, in themselves define the novel features of the diapers of this invention. Therefore, said descriptions are not to be interpreted as limitations on the nature or type of the layers used, but are merely referred to for the purpose of general construction of the diapers.

Referring back to the drawings, the insert 31 as shown in FIGS. 1-3 comprises a bodyside surface 37 which faces, or is in contact with the skin, and an opposed surface 39. At its distal end 33, the insert 31 is provided with a pair of opposed and spaced apart hooks 41 and 43 dimensioned to securely engage onto the loops 27,29 as hereinafter explained.

In order to wear the diaper, the belt 11 is wrapped around the torso of the user so that the hook 23 engages the loop 25. Since the belt is stretchable to various degrees, it may be in a relatively small size in order to realize substantial savings in materials used to define the landing zones. By stretching the belt 11 to a predetermined degree, it can be made to fit a given size torso and thus fit comfortably and tightly by engaging the hook 23 onto the loop 25. Thereafter, the hooks 41 and 43 are securely engaged onto the complementary loops 27 and 29, which are normally located on the opposite surface 19 of the belt 11 will be facing the hooks 41 and 43 in order to engage therein. As it can be further seen from FIG. 3, when the diaper 10 is assembled as aforesaid, it will define the leg regions 45 and 27 for inserting the legs of the wearer therethrough.

From the foregoing description of the diaper of this invention, it can be appreciated that relative locations of the hooks and loops on the belt 11 and insert 31 are critical to the proper assembly and advantageous functioning of the diaper. These, together with the degree of elasticity of the belt and the location of the elastic members are the principle features which contribute to the efficacy of the invention and improved performance of the diaper.

In a further preferred construction, the belt 11 is only partly elasticated as shown in FIG. 4. In this construction, the areas under the loops are preferably not elasticated. Also, the belt may be made of two pieces; one elasticated material and one non-elasticated which may be joined together by an adhesive or heat seal.

In the embodiment of the invention illustrated in FIGS. 1A, 2A and 3A, the relative locations of the hooks and loops are changed as shown in the drawings without, however, changing the structure of the absorbent article and its manner of use around the torso of the user. Thus, as seen from FIGS. 1A, 2A and 3A the belt 11A of the diaper 10A has a back waist portion 13A and a front waist portion 15A each having a bodyside surface 17A and an opposed surface 19A away from the bodyside surface. The bodyside surface 17A is provided with a male fastener, such as the hook member 23A, located at one lateral end of the belt, and a female fastener, such as the loop member 25A, located at the other lateral end of the belt. If desired, conventional adhesive tape tabs and landing zones may be used in lieu of the hook and loop members. Also, the relative positions of the male fastener 23A and the female fastener 25A may be reversed without affecting the structure and manner of assembly or use of the article. Furthermore, as shown in FIGS. 1A and 2A, the distal end 33A of the insert member 31A is provided with spaced apart female fasteners such as loop members 27A and 29A, and the male fasteners 41A, 43A. FIG. 3A shows the diaper of this embodiment in assembled position which, as it can be seen, is similar in construction to the diaper shown in FIG. 3. Once again, the hook and loop members in FIG. 1A may be replaced with male and female mechanical fasteners, tape tabs or adhesive tabs, if desired, without changing the structure or use of the absorbent article.

The insert member 31A has a bodyside surface 37A which faces, or is in contact with the skin, and an opposed surface 39A (see FIG. 3A). The proximal end 35A of the insert 31A is, during use, attached to the belt 11A. Thus, the belt 11A and insert 31A may be separate components which can be assembled when used, or they can form an integral combination ready to be worn by the user. As seen from FIG. 3A, when the diaper is assembled, it will define the leg regions 45A,47A for inserting the legs of the wearer therethrough.

In the embodiment of the invention illustrated in FIGS. 1A, 2A and 3A, one wing of the belts is longer than the other wing. As used herein, the term “wing” refers to that segment of the belt which lies on a side (left or right) of the vertical axis x-x that passes through the insert member 31A. Thus, the left wing is that segment of the belt which is to the left of the vertical axis x-x as viewed by the user and which spans the distance L₁, as shown in FIG. 1A, and the right wing is that segment of the belt which is to the right of the vertical axis x-x as viewed by the user and which spans the distance L₂ as shown in FIG. 1A. In FIG. 1A, the distance L₁ is greater than the distance L₂ in order to accommodate three fasteners. In a variation of this embodiment, the wing which contains three fasteners may be the right wing in which case the right wing will be longer than the left wing, i.e., L₂ will be greater than L₁. Preferably, the length of L₂ is from about 5 to about 95 percent the length of L₁ and is most preferably from about 20 to about 80 percent the length of L₁.

In the embodiment of the invention illustrated by FIG. 1A, the longer wing may be fully or partly elasticated by elastic members such as the elastic members 21A. The shorter wing may also be fully or partly elasticated but for cost reasons, it is preferably non-elastic or only partly elastic.

The materials and fabrics used in making the diaper 10A of FIG. 1A of the present invention are the same as those used in making the diaper illustrated in FIGS. 1-3. In use, the wings of the belt are wrapped around the torso of the wear and are secured in position by engaging the hook 23A and loop 25A disposed at the lateral ends of the belt 11A. The distal end 33A of the insert 31A is then folded over and the loops 27A and 29A are engaged with the hooks 41A and 43A, respectively. The assembled diaper is shown in FIG. 3A.

In the embodiment shown in FIG. 1B, the diaper 10B comprises the half-belt or a single wing 11B which may be partially elasticated by the elastic elements 21B. The half-belt 11B is attached to the insert member 31B which has a distal end 33B and a proximal end 35B to which the half-belt 11B is attached by sewing or by an adhesive. The term “half-belt” refers to a structure such as half the length of the belt 11A shown in FIG. 1A which, when attached to the proximal end of the insert member forms a single wing. Preferably, the half-belt 11B can be sandwiched between the cover sheet and the backsheet. The insert member 31B has a bodyside surface 37B which faces, or is in contact with the skin, and an opposed surface 39B.

The half-belt 11B is provided with three fasteners; one fastener 23B at the lateral end thereof away from the proximal end 35B of the insert member 31B, and two spaced apart fasteners 41B and 43B disposed between the fastener 23B and said proximal end 35B. The insert member is provided with a fastener 25B at the lateral edge thereof of the proximal end 35B, away from the belt 11B, and two spaced apart fasteners 27B and 29B at the distal end 33B. In one embodiment the fastener 23B may be a male fastener, such as a hook, and fastener 25B may be a female fastener, such as a loop. Each of the fasteners 41B, 43B may also be a male fastener such as a hook, and each of the fasteners 27B, 29B may be a female fastener such as loop. The fasteners 41B, 43B and 27B, 29B are located such that when the diaper is worn, the half-belt 11B is folded over the proximal end 35B thereby engaging the fastener 23B with the fastener 25B. The distal end 33B of the insert member 31B is then folded over onto the proximal end 35B thereby causing the fasteners 41B, 43B to engage with the correspondingly spaced fasteners 27B, 29B.

In lieu of the hook (male) and (female) loop, tape tabs and landing zones may be used to procure securement of the diaper, and the positions of the male and female fasteners may be altered with the same results. For example, fastener 23B may be a female fastener and fastener 25B may be a male fastener. Also, fasteners 41B, 43B may be female fasteners and the fasteners 27B, 29B may be male fasteners, if desired without altering the assembled diaper. Similarly, while the half-belt or wing 11B is shown to the left of the insert member as viewed from FIG. 1B, the half-belt 11B may be disposed to the right of the insert member, in which case the fastener 25B will be located at the other lateral edge of the proximal end 35B.

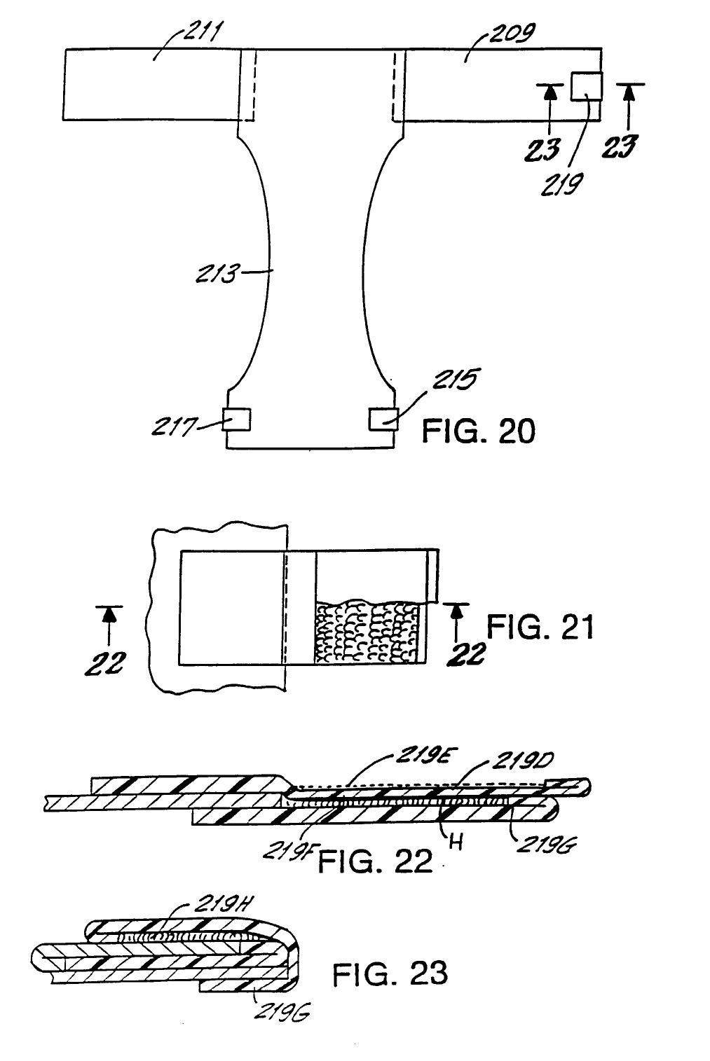

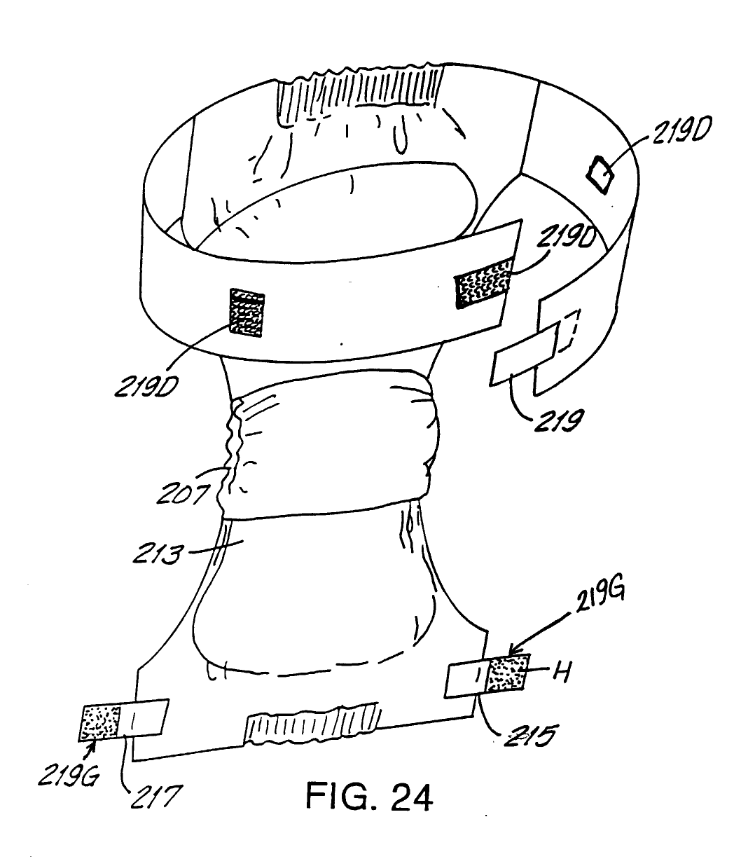

The disposable absorbent articles illustrated in FIGS. 1-5 and 1A-3A have three fasteners on one wing of the belt, one fastener at the proximal end of the insert member and two fasteners at the distal end of the insert member. The half-belted article shown in FIG. 1B also has three fasteners on the half-belt, a fastener at the lateral edge of the insert member and two fasteners at the distal end of the insert members. The fasteners are male and female fasteners and are selected and positioned such that when the diaper is assembled for wearing by the user, appropriate ones of the male/female fasteners engage with one another to secure the article around the torso of the user. It must be understood, however, that the fasteners which can be employed to achieve securement of the article are not limited to the fasteners described in connection with FIGS. 1-5, 1A-3A and 1B. Other fastening systems may be used such as the fastening systems described in the aforementioned copending application Ser. No. 09/844,726 filed Apr. 27, 2001 which is a continuation-in-part of said application Ser. No. 09/797,334, the disclosures of which are fully incorporated herein by reference.

A first fastening system (tape tabs and landing zone) is shown in FIGS. 6-9 and a diaper incorporating this fastening system is shown, in perspective view, in FIGS. 10-11. As shown in FIGS. 10 and 11, the diaper generally designated as 200 comprises a chassis having a back waist region 201 a front waist region 203 and a crotch region 205 and an absorbent core 207. A pair of opposed lateral segments or wings 209, 211 extend from the respective edges of the back waist region 201, and a generally vertical intersecting piece 213 having a proximal end 213A attached to the diaper chassis and a distal end 213B with a pair of opposed tapes 215, 217 disposed at each side of the intersecting piece near the distal end thereof.

Referring to FIGS. 6-9, there is shown in FIGS. 6 and 7 the tape tab 219 having a portion 219A used to fasten the tape to the diaper, and a second portion 219B permanently attached to the bottom surface of the wing 209, i.e., the surface away from the skin of the wearer (see FIGS. 8 and 9). The tape portion 219 has a top surface 221 covered with a pressure sensitive adhesive, and an opposed bottom surface made of a suitable plastic such as, e.g., polyethylene or polypropylene film, or other material such as, e.g., woven or nonwoven. As shown in FIG. 7, the fastening system comprises a release tape 223 having a top surface coated with a release agent such as a silicone compound, and an opposed bottom adhesive surface with a portion of the release tape 223 attached to the portion 219A and the other portion adhered to the top surface of the wing 209. Before use, the portion 219A is folded over the portion 219B in order to protect the adhesive surface during transportation of the product. For convenience of manipulation, the lateral edge of the tape 219 is folded upon itself so as to form an adhesive-free grip strip (finger lift) 225.

The other component of the fastening system are landing zones (tapes) 227,229 located on the lateral segments or wings 209 and 211, respectively. The size of each landing zone may be varied if desired depending on the size of the diaper. Each landing zone has an outer surface covered, at least partly, with a release agent such as a silicone compound. This allows the tape tab 219 to be positioned and repositioned on the landing zone 229 several times without tearing the diaper fabric.

In use, the tape tabs 219 is peeled away from the tape tab portion 219A by gripping and pulling away the grip strip 225. The diaper wings 209 and 211 are then wrapped around the waist of the wearer and the pressure sensitive surface 221 of the tape 219 is secured to the landing zone 229. Thereafter, the insert piece 213 is passed under the crotch, folded thereover and the tape tabs 215 and 217 located at the distal end of the insert piece is releasably secured to the landing zones such that tape tab 215 is secured to the landing zone 227 and tape tab 217 is secured to the landing zone 229. Tape tabs 215 and 217 may each have the same construction as tape tab 219, if desired. As it can be seen, this fastening system permits repeated adjustments and repositioning of the tape tabs on the landing zones to achieve a desired fit without tearing the fabric of the diaper.

The second fastening system is a prelaminated multi-layer tape tabs and landing zone shown in FIGS. 12-15 which is similar to the first fastening system illustrated in FIGS. 6-9 except for differences in the fastener 219 discussed below. As shown in FIG. 12 the T-shaped diaper 200 comprises a chassis having opposed lateral segments or wing 209,211. The construction of the diaper is otherwise the same as in FIG. 6. The fastener 219, however, consists of two layers of tapes 219A, 219C. Initially, the layer 219A is used to fasten the tape 219 to the diaper. In order to readjust the diaper, the layer 219C may be peeled off, leaving the layer 219A in place to act as a landing zone for readjustment of the diaper when necessary. After readjusting the diaper, the layer 219C is attached back onto layer 219A. Thus, the second fastening system requires less landing zone than the first system while still realizing the advantage of repeated readjustment of the diaper and repositioning of the tape tab without tearing the diaper fabric. The use of multi-layer tape tab according to this fastening system, with less landing zone area, results in increased flexibility of the wing portion of the diaper, and permits the use of elasticized wings when desired, all resulting in decreased manufacturing cost of the diaper. The construction of tape tabs 215,217 may be similar to tape tab 219.

The third fastening system employs hook and loop fastening system as illustrated in FIGS. 16-19. This fastening system is similar to the first fastening system described in connection with FIGS. 6-9 except that the landing zones 227 and 229 are covered with loop material rather than a silicone compound and the top surface of the tape portion 219A is covered with a hook material which covers the pressure sensitive adhesive. Thus, the release tab 223 covers the adhesive area that is not covered by the hook material. Referring to FIGS. 1619, it can be seen that the configuration and construction of the T-shaped diaper of FIG. 16 is the same as in FIG. 6. Instead of being covered by a pressure sensitive adhesive as in FIG. 7, the portion 219A is covered with layer of hook material H, e.g., Velcro®. A grip strip 225 facilitates gripping the end of the tape 219 when peeling the fastener.

In use, the diaper wings are wrapped as hereinbefore described, the grip strip 225 is gripped to peel the tape 219 away and expose the hook surface H and then attaching (engaging) the hook surface H onto the loop-landing zone 229 on the wing. The intersecting portion 213 is then passed under the crotch, folded thereover and the tabs 215,217 are secured to the landing zones 227,229, respectively. Again the construction of tape tabs 215,217 may be identical to tape tabs 219 in FIGS. 6, 12 and 16.

Referring to FIGS. 20-23 the fourth fastening system is a prelaminated multi-layer hook and loop tape tab and landing zone similar to the second system except that the tape 219 consists of two superposed layers; a layer 219D having a top adhesive surface 219E and an opposed bottom surface 219F covered with loop material. Superposed under the layer 219D is the layer 219G which is covered with hook material H, the same as layer 219A in FIG. 18. Again tape tabs 215 and 217 may be identical to tape tab 219.

FIG. 24 illustrates a diaper, in semi-assembled position, incorporating the fastening system as the second and fourth fastening systems. In FIG. 24, the tabs 215,217 are shown in open position after use of the diaper in order to further illustrate the fastening system. As it can be seem from this figure, after the diaper has been worn and the insert piece 213 has been removed to disassemble the diaper, there remains on the wings 227 and 229, the layers 219D as described in connection with FIG. 22.

The four fastening systems described in the aforementioned pending application Ser. No. 09/844,726 filed Apr. 27, 2001 and Ser. No. 09/797,334 filed Mar. 1, 2000 may be used in the embodiment of the invention shown in FIG. 1B. When used in this embodiment, the fastener 25B may be a tape tab having the same structure as the tape tab 219 shown in FIGS. 6-11. The remaining fasteners and this detailed description are basically the same as hereinbefore described in connection with FIGS. 12-24.

From the foregoing description of the structure of the diaper and its manner of assembly, it can be appreciated that the relative positions and locations of the fasteners can be changed without altering the manner of assembling the diaper and its construction.

While four fastening systems have been described, other forms of fastening systems may be employed such as hook and loop, tape tabs and landing zones. For example, instead of tape tab with hook and loop (third fastening system), one may use a patch of tape having an adhesive coating on its bottom side and hook or loop on its other side. Other changes and fastening systems means may be employed which are obvious from the foregoing detailed description and which are nevertheless within the scope of this invention.

1. A disposable absorbent article adapted to fit individuals having various torso sizes, said article, in use, comprising a belt and an insert member disposed vertically along a vertical axis relative to said belt when said belt is in stretched horizontal position, said insert member having a proximal end attached to said belt, and a distal end having two spaced apart fasteners,
said belt having a first lateral portion disposed on one side of the proximal end of said insert member and a second lateral portion disposed on the other side of the proximal end of said insert member, wherein the length of one of said lateral portions of said belt is from about 5 to about 95 percent the length of the other lateral portion of said belt,
a fastener disposed on the body side surface at one end of said belt when said belt is viewed in a generally stretched position; and a fastener disposed on the opposed surface of said belt at the other end of said belt, said fasteners being adapted to engage with each other when said first and second lateral portions of the belt are wrapped around the torso of a user;
a pair of spaced apart, generally parallel fasteners disposed on said longer portion of said belt member;
said proximal end of said insert member being attached to said belt and said distal end being adapted to be folded onto said proximal end when said article is worn by the user;
said pair of spaced apart fasteners disposed at the distal end of said insert member being complementary to and adapted to engage with said pair of fasteners on said belt when said belt is wrapped around the torso of the user and said distal end is folded over said proximal end of said insert member.

2. A disposable absorbent article adapted to fit individuals having various torso sizes, said article, in use, comprising a belt and an insert member disposed vertically along a vertical axis relative to said belt when said belt is in stretched horizontal position, said insert member having a proximal end attached to said belt, and a distal end having two spaced apart tape tab fasteners,
said belt having a first lateral portion disposed on one side of the proximal end of said insert member and a second lateral portion disposed on the other side of the proximal end of said insert member, wherein the length of one of said lateral portions of said belt is from about 5 to about 95 percent the length of the other lateral portion of said belt,
a tape tab disposed at the lateral end of one of the lateral portions of said belt, a first landing zone disposed at the lateral end of the other lateral portion of said belt adapted to engage with said tape tab when said lateral portions are wrapped around the waist of a wearer, and two additional landing zones on said longer lateral portion of said belt member, said two additional landing zones being spaced apart relative to each other,
said proximal end of said insert member being attached to said belt and said distal end being adapted to be folded onto said proximal end when said article is worn by the user;
said pair of spaced apart tape tabs disposed at the distal end of said insert member being complementary to and adapted to engage with said pair of additional spaced apart landing zones on said belt when said belt is wrapped around the torso of the user and said distal end is folded over said proximal end of said insert member,
each of said lateral tape tabs having a first portion adapted to be fastened to said landing zones and a second portion attached permanently to a surface of said lateral portions of said belt and distal end of said insert member away from the skin of the wearer of said article,
said landing zones having a top surface coated with a release compound and an opposed bottom adhesive surface, wherein the top surface of each landing zone is attached to said first portion of said tape tab, the bottom surface of said release tape is adhered to a lateral portion of said belt member.

3. A disposable absorbent article as in claim 2 wherein said tape tab is a pre-laminated tape tab having a portion consisting of two layers, a first layer having a top adhesive surface and a second layer having a top adhesive surface releasably adhered to said top layer, and wherein said second layer is initially releasably adhered to said first layer but is peelable therefrom to adjust use of the absorbent article.

4. A disposable absorbent article as in claim 2 wherein each of the landing zones on said belt are partly covered with loop material and wherein the top surface of said tape tabs are partly covered with hook material.

5. A disposable absorbent article as in claim 3 wherein each of said tape tabs is a pre-laminated hook and loop tape tab having two superposed layers, a first layer having a top adhesive surface, and an opposed bottom surface covered with loop material, and a second layer having a top surface covered partly with a hook material.

6. A disposable absorbent article as in claim 1 wherein said belt is partly stretchable.

7. A disposable absorbent article as in claim 2 wherein said belt is partly stretchable.

8. A disposable absorbent article as in claim 3 wherein said belt is partly stretchable.

9. A disposable absorbent article as in claim 4 wherein said belt is partly stretchable.

10. A disposable absorbent article as in claim 5 wherein said belt is partly stretchable.

11. A disposable absorbent article adapted to fit individuals of various torso sizes, said article, in use, comprising, in combination, a half-belt member and an absorbent insert member having a proximal end and a distal end;
said half-belt member having two lateral ends; a free lateral end and a second lateral end attached to the proximal end of said insert member, said half-belt having a body side surface and an opposed surface on the reverse side of said body side surface;
three fasteners disposed on said half-belt; one fastener at said free lateral end and two spaced apart generally parallel fasteners between said fastener and the proximal end of said insert member;
three fasteners disposed on said insert member, wherein one fastener is disposed at the lateral edge of the proximal end of said insert member adapted to engage with the fastener at the free end of said half-belt member when said half-belt member is wrapped around the torso of the wearer, and two spaced apart fasteners disposed at the distal end of said insert member adapted to engage with said spaced apart fasteners on said half-belt member when said distal end is folded onto said proximal end of said insert member during wear.

12. A disposable absorbent article adapted to fit individuals of various torso sizes, said article, in use, comprising, in combination, a half-belt member and an absorbent insert member having a proximal end and a distal end;
said half-belt member having two lateral ends; a free lateral end and a second lateral end attached to the proximal end of said insert member, said half-belt having a body side surface and an opposed surface on the reverse side of said body side surface;
three fasteners disposed on said half-belt; one tape tab fastener at said free lateral end and two spaced apart generally parallel landing zones fasteners between said tape tab fastener and the proximal end of said insert member;
three fasteners disposed on said insert member, wherein one tape tab fastener is disposed at the lateral edge of the proximal end of said insert member adapted to engage with the tape tab fastener at the free end of said half-belt member when said half-belt member is wrapped around the torso of the wearer, and two spaced apart tape tab fasteners disposed at the distal end of said insert member adapted to engage with said spaced apart landing zone fasteners on said half-belt member when said distal end is folded onto said proximal end of said insert member during wear.

13. A disposable absorbent article as in claim 12 wherein said tape tab is a pre-laminated tape tab having a portion consisting of two layers, a first layer having a top adhesive surface and a second layer having a top adhesive surface releasably adhered to said top layer, and wherein said second layer is initially releasably adhered to said first layer but is peelable therefrom to adjust the absorbent article.

14. A disposable absorbent article as in claim 12 wherein each of the landing zones on said belt are partly covered with loop material and wherein the top surface of said tape tabs are partly covered with hook material.

15. A disposable absorbent article as in claim 13 wherein each of said tape tabs is a pre-laminated hook and loop tape tab having two superposed layers, a first layer having a top adhesive surface, and an opposed bottom surface covered with loop material, and a second layer having a top surface covered partly with a hook material.

16. A disposable absorbent article as in claim 11 wherein said half-belt member is partly stretchable.

17. A disposable absorbent article as in claim 12 wherein said half-belt member is partly stretchable.

18. A disposable absorbent article as in claim 13 wherein said half-belt member is partly stretchable.

19. A disposable absorbent article as in claim 14 wherein said half-belt member is partly stretchable.

20. A disposable absorbent article as in claim 15 wherein said half-belt member is partly stretchable.
